(19) United States
(12) Patent Application Publication
(10) Pub. No.: US 2025/0319051 A1
(43) Pub. Date: Oct. 16, 2025

(54) TUMOR CELL-DERIVED MICROPARTICLE LOADED WITH SUCCINIC ACID, PREPARATION METHOD THEREFOR, AND USE THEREOF

(71) Applicant: Huazhong University of Science and Technology, Wuhan (CN)

(72) Inventors: Ke Tang, Wuhan (CN); Bo Huang, Wuhan (CN)

(21) Appl. No.: 18/855,629

(22) PCT Filed: Apr. 3, 2023
(86) PCT No.: PCT/CN2023/085972

§ 371 Date: Oct. 9, 2024

(30) Foreign Application Priority Data
Apr. 11, 2022 (CN) 202210375748.4

(51) Int. Cl. A61K 31/194; A61K 9/51; A61K 47/46; A61P 35/00; B82Y 5/00

(52) U.S. Cl. CPC A61K 31/194; A61K 9/5176; A61K 47/46; A61P 35/00; B82Y 5/00

(57) ABSTRACT

The present disclosure relates to the technical field of biopharmaceuticals, and in particular to a tumor cell-derived microparticle loaded with succinic acid, a preparation method therefor, and use thereof. Provided is a tumor cell-derived microparticle loaded with succinic acid, which includes a tumor cell-derived microparticle and succinic acid loaded in the tumor cell-derived microparticle. Experimental data show that compared with the tumor cell-derived microparticle or succinic acid, the provided tumor cell-derived microparticle loaded with succinic acid has a significant therapeutic effect on various subcutaneous tumors in mice, thereby prolonging the survival time of the mice. The tumor cell-derived microparticle, as a drug carrier, has higher safety and more abundant sources, and is convenient for actual production operations. Furthermore, provided is a preparation method for the tumor cell-derived microparticle loaded with succinic acid, which greatly improves the drug-loading capacity of the microparticle.

### CROSS-REFERENCE TO RELATED APPLICATION

The present application claims the priority to and the benefit of Chinese Patent Application No. 202210375748.4 filed with the Patent Office of CNIPA on Apr. 11, 2022 and entitled “TUMOR CELL-DERIVED MICROPARTICLE LOADED WITH SUCCINIC ACID, PREPARATION METHOD THEREFOR, AND USE THEREOF”, the entire contents of which are incorporated herein by reference.

### TECHNICAL FIELD

The present disclosure pertains to the field of bio-medical technology, and relates to preparation of an organic compound drug-loaded microparticle and use thereof, specifically to a tumor cell-derived microparticle loaded with succinic acid, a preparation method therefor, and use thereof.

### BACKGROUND

In recent years, the incidence of tumors has increased year by year with an obvious younger-age tendency. Tumor treatment has increasingly become people's major concern. The clinical tumor treatment is currently predominated by radiotherapy and chemotherapy, but this treatment method not only kills tumor cells, but also causes irreversible damage to patients themselves. As more and more chemotherapeutics are used, the drug resistance of tumor cells has also become a problem that cannot be ignored. It is of great urgency to find new tumor treatments.

The immune system can maintain the body healthy by recognizing and eliminating tumor cells in the tumor microenvironment. In order to be able to survive in the body, the tumor cells make the human immune system suppressed by different strategies to protect themselves from being killed normally, so that the tumor cells can survive at all stages of the antitumor immune response. Tumor immunotherapy is a therapeutic approach to control and eliminate tumors by restarting and maintaining the tumor-immune cycle to restore the body's normal antitumor immune response. In recent years, the tumor immunotherapy has exhibited a powerful antitumor activity in the treatment of a variety of tumors, e.g. solid tumors such as melanoma, non-small cell lung cancer, kidney cancer, and prostate cancer. Currently available products for tumor immunotherapy include monoclonal antibody-based immune checkpoint inhibitors, therapeutic antibodies, cancer vaccines, cell therapy and small molecule inhibitors, etc.

As reported in Cited Literature 1 and Cited Literature 2, the tumor cell-derived microparticles can induce a tumor immune response. The tumor cell-derived microparticles are secreted by the tumor itself, and can be recognized and phagocytosed by tumor cells. At present, there have been some studies on the tumor cell-derived microparticles, for example, Cited Literature 3 discloses integrating functional inorganic nanoparticles with cell-derived microparticles and encapsulating a chemotherapeutic therein to achieve the effect of treating cancer.

In another aspect, the tumor microenvironment is a complex environment on which tumor cells depend for survival, and composed substantially of a variety of different extracellular matrices and stromal cells. Tumor-associated macrophages are macrophages infiltrated in tumor tissue and are the most abundant immune cells in the tumor microenvironment. Studies have shown that tumor-associated macrophages can promote the growth and metastasis of tumor cells through multiple pathways, and they promote tumor growth by regulating the metabolism of tumor cells. Studies have found that succinic acid, as a vital metabolite of the tricarboxylic acid cycle, can promote the release of inflammatory factors. However, succinic acid cannot freely enter cells to exert its effects because it is a water-soluble organic compound. As reported in Cited Literature 4, cancer cells can polarize macrophages into tumor-associated macrophages by releasing succinic acid into the tumor microenvironment, thereby promoting tumor growth and metastasis.

### CITED LITERATURES

Cited Literature 1: Zhang HF. Mechanism of tumor cells derived microparticles mediated antitumor immune response [D]. Huazhong University of Science and Technology, 2012.

Cited Literature 2: Zhang H, Tang K, Zhang Y, Ma R, Ma J, Li Y, Luo S, Liang X, Ji T, Gu Z, Lu J, He W, Cao X, Wan Y, Huang B. Cell-free tumor microparticle vaccines stimulate dendritic cells via cGAS/STING signaling [J]. Cancer Immunol Res. 2015, 3(2):196-205.

Cited Literature 3: CN109771376A

Cited Literature 4: Wu JY, Huang TW, Hsieh YT, Wang YF, Yen CC, Lee GL, Yeh CC, Peng YJ, Kuo YY, Wen HT, Lin HC, Hsiao CW, Wu KK, Kung HJ, Hsu YJ, Kuo CC. Cancer-Derived Succinate Promotes Macrophage Polarization and Cancer Metastasis via Succinate Receptor [J]. Mol Cell. 2020 Jan. 16; 77(2):213-227.e5.

### SUMMARY

### Technical Problem

In view of the above problems existing in the prior arts, the present disclosure intends to provide a tumor cell-derived microparticle loaded with succinic acid, and provide a preparation method therefor and use thereof, so as to provide a safer and more effective method reference for tumor treatments, while broadening the scope of applications of the tumor cell-derived microparticles and succinic acid.

### Solution to Problem

[1]. A tumor cell-derived microparticle loaded with succinic acid, comprising a tumor cell-derived microparticle and succinic acid loaded therein.
[2]. The tumor cell-derived microparticle loaded with the succinic acid according to [1], wherein the tumor cell is a solid tumor cell or a nonsolid tumor cell; preferably, the tumor cell-derived microparticle has a particle size of 50 to 500 nm.
[3]. The tumor cell-derived microparticle loaded with the succinic acid according to [1] or [2], wherein a ratio of a number of the tumor cell-derived microparticle to a content of the succinic acid is 1×10⁹ to 1×10¹¹: 25 to 60 ng.
[4]. A method for preparing a tumor cell-derived microparticle loaded with succinic acid, comprising the following steps:
   (i) inducing apoptosis of a tumor cell to release a tumor cell-derived microparticle, mixing the tumor cell-derived microparticle with succinic acid to obtain a mixture I, and subjecting the mixture I to electro-transformation and centrifugation to obtain the tumor cell-derived microparticle loaded with the succinic acid; or
   (ii) mixing a tumor cell with succinic acid to obtain a mixture II, co-incubating the mixture II, inducing apoptosis of the tumor cell in the mixture II to release a tumor cell-derived microparticle loaded with the succinic acid, and obtaining the tumor cell-derived microparticle loaded with the succinic acid after centrifugation.
[5]. The method according to [4], wherein a number of the tumor cell in the mixture II is 1×10⁷ to 1×10⁸; a number of the tumor cell-derived microparticle in the mixture I is 1×10¹⁰ to 1×10¹¹; and the succinic acid in the mixture I or the mixture II has a concentration of 0.5 to 2 mM.
[6]. The method according to [4] or [5], wherein the inducing apoptosis of the tumor cell is conducted under conditions of suspending the tumor cell in normal saline and leaving at 37° C. for 20 to 30 h; and the centrifugation is conducted under conditions of centrifuging at 500 to 50000 g for 1 to 1.5 h.
[7]. The method according to [4] or [5], wherein the electro-transformation is conducted under the following conditions: 150 to 300 V of voltage; 100 to 200 μF of capacitance; 3 to 5 ms of pulse time; and 1 to 6 times of electroporation.
[8]. The method according to [4] or [5], wherein the co-incubation is conducted under conditions of leaving at 37° C. for 20 to 30 h.
[9]. Use of the tumor cell-derived microparticle loaded with the succinic acid according to any one of [1] to [3] and/or a tumor cell-derived microparticle loaded with succinic acid prepared by the method according to any one of [4] to [8] in a preparation of a medicament for preventing and/or treating cancer.
[10]. The use according to [9], wherein the cancer comprises liver cancer, bladder cancer, bone cancer, brain cancer, breast cancer, colorectal cancer, endometrial cancer, cervical cancer, esophageal cancer, eye cancer, head and neck cancer, kidney cancer, lung cancer, gallbladder cancer, melanoma, ovarian cancer, pancreatic cancer, prostate cancer, gastric cancer, thyroid cancer, leukemia, lymphoma, and myeloma.
[11]. A method for preventing and/or treating cancer, comprising administering, to an individual in need thereof, an effective dose of the tumor cell-derived microparticle loaded with the succinic acid according to any one of [1] to [3] and/or a tumor cell-derived microparticle loaded with succinic acid prepared by the method according to any one of [4] to [8]; preferably, the cancer comprises liver cancer, bladder cancer, bone cancer, brain cancer, breast cancer, colorectal cancer, endometrial cancer, cervical cancer, esophageal cancer, eye cancer, head and neck cancer, kidney cancer, lung cancer, gallbladder cancer, melanoma, ovarian cancer, pancreatic cancer, prostate cancer, gastric cancer, thyroid cancer, leukemia, lymphoma, and myeloma.

### Advantageous Effects of the Invention

Tumor cell-derived microparticles are endogenous substances of the body, which themselves can be better recognized and ingested by tumor cells. In view of this, the tumor cell-derived microparticles when used as a drug carrier are safer, richer in source, and readily accessible as compared to activation by an antigen alone or other immune drugs, and they can target a vast majority of tumors and infections and thus have good universality. The tumor cell-derived microparticles loaded with succinic acid provided herein are used as drug-loaded microparticles. Their preparation method allows for a higher drug-loading capacity, and the loaded drug further expands the treated type and scope of the disease, reduces the cost and risk, and renders the operation more concise. The experimental data show that compared with the tumor cell-derived microparticles or succinic acid per se, the tumor cell-derived microparticles loaded with succinic acid provided herein have a significant therapeutic effect on a variety of subcutaneous tumors in mice and improve the survival time of mice.

### BRIEF DESCRIPTION OF THE DRAWINGS

FIG. 1A and FIG. 1B show the particle size and morphology of tumor cell-derived microparticles, respectively.

FIG. 2 shows the concentration of succinic acid loaded in tumor cell microparticles.

FIGS. 3A, 3B, and 3C show the tumor morphology, tumor volume, and survival rate of mice after treatment of a mouse subcutaneous tumor model of breast cancer, respectively.

FIGS. 4A and 4B show the number of abdominal tumor nodules after treatment in a mouse colorectal cancer model; FIGS. 4C and 4D show the tumor weight and the survival rate of mice after treatment in a mouse colorectal cancer model, respectively.

FIGS. 5A, 5B, 5C, and 5D show the tumor morphology, tumor weight, tumor volume, and body weight of mice after treatment in a mouse subcutaneous tumor model of liver cancer, respectively.

FIGS. 6A and 6B show uptake of tumor cell microparticles by different immune cells.

FIG. 7 shows co-localization of macrophages and tumor cell microparticles.

### DETAILED DESCRIPTION

The embodiments of the present disclosure will be illustrated below, but the present disclosure is not limited thereto.

In the present disclosure, the term “may” involves both the meaning of doing something and the meaning of not doing something.

In the present disclosure, the term “optional” or “optionally” means that the event or situation described subsequently may or may not occur, and the description includes the situation where the event occurs and the situation where the event does not occur.

In the present disclosure, the term “including”, “having”, “comprising” or “containing” may be intended to be inclusive or open-ended, and does not exclude additional or unrecited elements or methods and steps. Meanwhile, the term “including”, “having”, “comprising” or “containing” may also be intended to be close-ended and excludes additional or unrecited elements or methods and steps.

The numerical range represented by “numerical value A to numerical value B” or “numerical value A-numerical value B” used in the present disclosure refers to the range including the endpoint values A and B.

In the present disclosure, the terms “tumor cell microparticle”, “tumor cell-derived microparticle”, “microparticle derived from tumor cell”, and “microparticle of tumor cell source” are used interchangeably and refer to cell vesicles produced due to apoptosis of tumor cells, which are not loaded with any pharmaceutical ingredient.

In the present disclosure, the term “succinic acid” refers to succinic acid or a pharmaceutically acceptable salt thereof.

In the present disclosure, the term “individual”, “object”, “patient” or “subject” includes mammals. The mammals include, but are not limited to, domesticated animals (e.g. cattle, sheep, cats, dogs or horses), primates (e.g. humans or non-human primates such as monkeys or orangutans), rabbits, and rodents (e.g. mice, rats or guinea pigs).

In the present disclosure, the term “tumor cell” may be a cell in a solid tumor (including a cell having the potential or ability to form a solid tumor) or a cell in a nonsolid tumor.

In the present disclosure, the term “about” may mean that a value includes the standard deviation of the error caused by the apparatus or method used to measure the value. Unless otherwise expressly stated, it shall be appreciated that all ranges, numbers, numerical values, and percentages used herein have been modified by the term “about”.

### Tumor Cell-Derived Microparticle Loaded with Succinic Acid

The present disclosure provides a tumor cell-derived microparticle loaded with succinic acid, comprising a tumor cell-derived microparticle and succinic acid loaded therein.

In some embodiments, the tumor cell is a solid tumor cell or a nonsolid tumor cell. Accordingly, the tumor cell-derived microparticle is a solid tumor cell-derived microparticle or a nonsolid tumor cell-derived microparticle. Furthermore, in some preferred embodiments, the tumor cell includes, but is not limited to, tumor cells in liver cancer, bladder cancer, bone cancer, brain cancer, breast cancer, colorectal cancer, endometrial cancer, cervical cancer, esophageal cancer, eye cancer, head and neck cancer, kidney cancer, lung cancer, gallbladder cancer, melanoma, ovarian cancer, pancreatic cancer, prostate cancer, gastric cancer, thyroid cancer, leukemia, lymphoma, myeloma, etc. In some specific embodiments, the tumor cell is a melanoma cell, a lung cancer cell, a leukemia cell or a liver cancer cell, preferably a liver cancer cell.

In some embodiments, the tumor cell-derived microparticle has a particle size of 50 to 500nm, preferably 100 to 300 nm, more preferably 120 to 250 nm.

In some embodiments, the ratio of the number of the tumor cell-derived microparticles to the content of the succinic acid is 1×10⁹ to 1×10¹¹: 25 to 60 ng, preferably 1×10¹⁰: 25 to 60 ng, preferably 1×10¹⁰: 30 to 60 ng, more preferably 1×10¹⁰: 35 to 60 ng, further preferably 1×10¹⁰: 40 to 60 ng, even more preferably 1×10¹⁰: 55 ng.

### Preparation Method for Tumor Cell-Derived Microparticle Loaded with Succinic Acid

The present disclosure provides a method for preparing the above-mentioned tumor cell-derived microparticle loaded with the succinic acid, comprising an electro-transformation method and a co-incubation method.

### Electro-Transformation Method

In some embodiments, the electro-transformation method comprises the following steps: inducing apoptosis of a tumor cell to release a tumor cell-derived microparticle, mixing the tumor cell-derived microparticle with succinic acid to obtain a mixture I, and subjecting the mixture I to electro-transformation and centrifugation to obtain the tumor cell-derived microparticle loaded with the succinic acid.

In some embodiments, the tumor cell is a solid tumor cell or a nonsolid tumor cell. In some preferred embodiments, the tumor cell includes, but is not limited to, tumor cells in liver cancer, bladder cancer, bone cancer, brain cancer, breast cancer, colorectal cancer, endometrial cancer, cervical cancer, esophageal cancer, eye cancer, head and neck cancer, kidney cancer, lung cancer, gallbladder cancer, melanoma, ovarian cancer, pancreatic cancer, prostate cancer, gastric cancer, thyroid cancer, leukemia, lymphoma, myeloma, etc. In some specific embodiments, the tumor cell is a melanoma cell, a lung cancer cell, a leukemia cell or a liver cancer cell, preferably a liver cancer cell.

In some embodiments, the method for inducing apoptosis of the tumor cell is starvation treatment. It is preferred that the tumor cell is subjected to the starvation treatment in normal saline under the specific conditions of suspending the tumor cell in normal saline, and leaving it at 37° C. for 20 to 30 h, preferably for 24 h.

In some embodiments, after inducing apoptosis of the tumor cell to release tumor cell-derived microparticles, the microparticles are collected. The specific collection method may be as follows: the cell suspension is collected and centrifuged at 500 to 1000 g for 3 to 7 min, preferably centrifuged at 800 g for 5 min; the supernatant is collected and subjected to gradient centrifugation at 1000 to 50000 g for 1 to 1.5 h, and a pellet is observed, which is the tumor cell-derived microparticle. The preferred specific steps of the gradient centrifugation are as follows: the supernatant is centrifuged at 15000 rpm for 6 min to obtain supernatant; the supernatant is collected and centrifuged at 5000 rpm for 10 to 15 min to obtain supernatant; the supernatant is collected and centrifuged at 14000 g for 5 min to obtain supernatant; and the supernatant is collected and centrifuged at 4° C. and 16000 g for 1 h. In some embodiments, when the number of tumor cells induced to apoptosis is 1×10⁷ to 1×10⁸, it is possible to harvest 1×10¹⁰ to 1×10¹¹ tumor cell-derived microparticles.

In order to improve the succinic acid-loading efficiency of the tumor cell-derived microparticles, in some embodiments, the number of the tumor cell-derived microparticles in the above mixture I is 1×10¹⁰ to 1×10¹¹, preferably 1×10¹⁰; the concentration of the succinic acid in the mixture I is 0.5 to 2 mM, preferably 0.5 to 1.5 mM, more preferably 1 mM. In some embodiments, in order to adapt to the above concentration of the succinic acid in the mixture I, the mass of the succinic acid in the mixture I may be 100 to 500 μg, e.g. 100 to 472 μg, 118 to 500 μg, 118 to 472 μg, 100 to 354 μg, 118 to 354 μg, 118 μg, 236 μg, 354 μg, or 472 μg.

In some embodiments, the conditions for the above electro-transformation are as follows: 150 to 300 V, preferably 270 V of voltage; 100 to 200 μF, preferably 150 μF of capacitance; 3 to 5 ms, preferably 4 ms of pulse time; and 1 to 6 times, preferably 3 times of electroporation.

In some embodiments, the conditions for the centrifugation are as follows: centrifuging at 500 to 50000 g for 1 to 1.5 h, preferably centrifuging at 16000 g for 1 h, preferably centrifuging at a low-temperature environment (4° C.).

### Co-Incubation Method

In some embodiments, the co-incubation method comprises the following steps: mixing a tumor cell with succinic acid to obtain a mixture II, co-incubating the mixture II, inducing apoptosis of the tumor cell in the mixture II to release a tumor cell-derived microparticle loaded with the succinic acid, and obtaining the tumor cell-derived microparticle loaded with the succinic acid after centrifugation.

In some embodiments, the tumor cell is a solid tumor cell or a nonsolid tumor cell. In some preferred embodiments, the tumor cell includes, but is not limited to, tumor cells in liver cancer, bladder cancer, bone cancer, brain cancer, breast cancer, colorectal cancer, endometrial cancer, cervical cancer, esophageal cancer, eye cancer, head and neck cancer, kidney cancer, lung cancer, gallbladder cancer, melanoma, ovarian cancer, pancreatic cancer, prostate cancer, gastric cancer, thyroid cancer, leukemia, lymphoma, myeloma, etc. In some specific embodiments, the tumor cell is a melanoma cell, a lung cancer cell, a leukemia cell or a liver cancer cell, preferably a liver cancer cell.

In order to improve the succinic acid-loading efficiency of the tumor cell-derived microparticles, in some embodiments, the number of the tumor cells in the above mixture II is 1×10⁷ to 1×10⁸, preferably 1×10⁷; the concentration of succinic acid in the mixture II is 0.5 to 2 mM, preferably 0.5 to 1.5 mM, more preferably 1 mM. In some embodiments, in order to adapt to the above concentration of succinic acid in the mixture II, the mass of succinic acid in the mixture II may be 100 to 500 μg, e.g. 100 to 472 μg, 118 to 500 μg, 118 to 472 μg, 100 to 354 μg, 118 to 354 μg, 118 μg, 236 μg, 354 μg, or 472 μg.

In some embodiments, the co-incubation is conducted under the conditions of leaving at 37° C. for 20 to 30 h, preferably for 24 h.

In some embodiments, the method for inducing apoptosis of the tumor cell is the same as that described in the above electro-transformation method. In some embodiments, after inducing apoptosis of the tumor cells in the mixture II to release microparticles, the collection method for the microparticles are the same as that described in the above electro-transformation method. In some embodiments, when the number of tumor cells induced to apoptosis is 1×10⁷ to 1×10⁸, it is possible to harvest 1×10¹⁰ to 1×10¹¹ tumor cell-derived microparticles loaded with succinic acid.

### Medical Use

The tumor cell-derived microparticles loaded with succinic acid provided herein can be used for preventing and/or treating cancer; preferably, the cancer includes liver cancer, bladder cancer, bone cancer, brain cancer, breast cancer, colorectal cancer, endometrial cancer, cervical cancer, esophageal cancer, eye cancer, head and neck cancer, kidney cancer, lung cancer, gallbladder cancer, melanoma, ovarian cancer, pancreatic cancer, prostate cancer, gastric cancer, thyroid cancer, leukemia, lymphoma, and myeloma; more preferably, the cancer is liver cancer, breast cancer, and colorectal cancer.

Tumor cell-derived microparticles loaded with succinic acid prepared by the method for preparing a tumor cell-derived microparticle loaded with succinic acid as provided herein can be used for preventing and/or treating cancer; preferably, the cancer includes liver cancer, bladder cancer, bone cancer, brain cancer, breast cancer, colorectal cancer, endometrial cancer, cervical cancer, esophageal cancer, eye cancer, head and neck cancer, kidney cancer, lung cancer, gallbladder cancer, melanoma, ovarian cancer, pancreatic cancer, prostate cancer, gastric cancer, thyroid cancer, leukemia, lymphoma, and myeloma; more preferably, the cancer is liver cancer, breast cancer, and colorectal cancer.

The present disclosure provides use of the above-mentioned tumor cell-derived microparticles loaded with succinic acid in the preparation of a medicament for preventing and/or treating cancer; preferably, the cancer includes liver cancer, bladder cancer, bone cancer, brain cancer, breast cancer, colorectal cancer, endometrial cancer, cervical cancer, esophageal cancer, eye cancer, head and neck cancer, kidney cancer, lung cancer, gallbladder cancer, melanoma, ovarian cancer, pancreatic cancer, prostate cancer, gastric cancer, thyroid cancer, leukemia, lymphoma, and myeloma; more preferably, the cancer is liver cancer, breast cancer, and colorectal cancer.

Use of tumor cell-derived microparticles loaded with succinic acid prepared by the method for preparing a tumor cell-derived microparticle loaded with succinic acid as provided herein in the preparation of a medicament for preventing and/or treating cancer; preferably, the cancer includes liver cancer, bladder cancer, bone cancer, brain cancer, breast cancer, colorectal cancer, endometrial cancer, cervical cancer, esophageal cancer, eye cancer, head and neck cancer, kidney cancer, lung cancer, gallbladder cancer, melanoma, ovarian cancer, pancreatic cancer, prostate cancer, gastric cancer, thyroid cancer, leukemia, lymphoma, and myeloma; more preferably, the cancer is liver cancer, breast cancer, and colorectal cancer.

The present disclosure provides a method for preventing and/or treating cancer, comprising administering, to an individual in need thereof, an effective dose of the tumor cell-derived microparticle loaded with the succinic acid; preferably, the cancer includes liver cancer, bladder cancer, bone cancer, brain cancer, breast cancer, colorectal cancer, endometrial cancer, cervical cancer, esophageal cancer, eye cancer, head and neck cancer, kidney cancer, lung cancer, gallbladder cancer, melanoma, ovarian cancer, pancreatic cancer, prostate cancer, gastric cancer, thyroid cancer, leukemia, lymphoma, and myeloma; more preferably, the cancer is liver cancer, breast cancer, and colorectal cancer.

The present disclosure provides a method for preventing and/or treating cancer, comprising administering, to an individual in need thereof, an effective dose of a tumor cell-derived microparticle loaded with succinic acid prepared by the method for preparing the tumor cell-derived microparticle loaded with succinic acid as provided herein; preferably, the cancer includes liver cancer, bladder cancer, bone cancer, brain cancer, breast cancer, colorectal cancer, endometrial cancer, cervical cancer, esophageal cancer, eye cancer, head and neck cancer, kidney cancer, lung cancer, gallbladder cancer, melanoma, ovarian cancer, pancreatic cancer, prostate cancer, gastric cancer, thyroid cancer, leukemia, lymphoma, and myeloma; more preferably, the cancer is liver cancer, breast cancer, and colorectal cancer.

### EXAMPLES

The present disclosure will be further illustrated with reference to the following examples, but any example or a combination thereof shall not be construed as a limitation on the scope or embodiments of the present disclosure. Experimental methods for which no specific conditions are indicated in the following examples are generally carried out in accordance with conventional conditions or those recommended by the manufacturers. In addition, any methods and materials similar or equivalent to those described herein can be applied to the methods of the present disclosure. Unless otherwise defined, all technical and scientific terms used herein have the same meanings as commonly understood by one of ordinary skill in the art. All reagents, materials or instruments are commercially available unless otherwise specified.

### Example 1: Preparation, Particle Size Detection and Electron Microscopy Detection of Tumor Cell-Derived Microparticles

Mouse liver cancer cells H22 in the logarithmic growth phase were collected and counted, and inoculated into the abdominal cavities in BALB/c mice at 1×10⁵ cells/mouse. One week later, the ascites cells were collected from the mice, counted, re-suspended in normal saline, and plated into a 15-cm cell culture dish at 1×10⁸ cells/dish. After the cell culture dish was placed in a cell incubator at 37° C. for 24 h, the cells were collected and centrifuged at 800 g for 5 min. The supernatant was collected and subjected to gradient centrifugation at 1000 to 50000 g for 1 to 1.5 h (the gradient centrifugation was carried out according to the following specific steps: the supernatant was centrifuged at 15000 rpm for 6 min to obtain supernatant; the supernatant was collected and centrifuged at 5000 rpm for 10 to 15 min to obtain supernatant; the supernatant was collected and centrifuged at 14000 g for 5 min to obtain supernatant; the supernatant was collected and centrifuged at 4° C. and 16000 g for 1 h to obtain pellets, which was the cell microparticles). The tumor cell microparticles were collected. The supernatant was discarded. The microparticles (visible pellets) were re-suspended in PBS and preserved at 4° C. 1×10⁷ tumor cells could produce about 1×10¹⁰ tumor cell-derived microparticles. The particle size of the microparticles was detected by the Nanoparticle Tracking Analysis (NTA), and the morphology of the microparticles was detected by the electron microscopy.

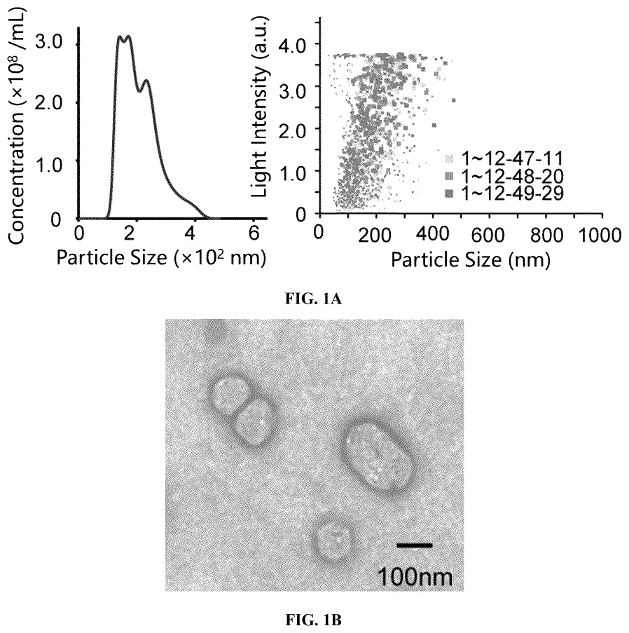

The results were as shown in FIG. 1A and FIG. 1B, showing the particle size and morphology of the tumor cell-derived microparticles. FIG. 1A showed the particle sizes of the tumor cell-derived microparticles detected by a nanoparticle tracker, from which it was clear that the particle sizes of the tumor cell-derived microparticles were uniformly distributed and concentrated at about 150 nm. FIG. 1B demonstrated the morphology of the tumor cell-derived microparticles observed by a scanning electron microscope, from which it was clear that the morphology of the tumor cell-derived microparticles was homogeneous, and their particle size distribution was consistent with the detection result obtained by the nanoparticle tracker.

### Example 2: Concentration of Succinic Acid Loaded in Tumor Cell Microparticles

Depending on the tumor cell microparticles obtained in Example 1, succinic acid was added at different concentrations (0.5 to 2 mM) to 1×10¹⁰ tumor cell microparticles. Preferably, the concentration of succinic acid was 1 mM. The mixed solution (about 2 mL) was added into an electroporation cuvette. The parameters of the electroporator were set (exponential wave: 270 V, 150 μF, 4 ms) for electroporation. The electroporation was conducted three times and the electroporation cuvette was put on a super clean bench quickly. The mixed solution was transferred into a 1.5-mL EP tube, and centrifuged at 16000 g and at 4° C. for 1 h. The supernatant was discarded. The tumor cell microparticles loaded with succinic acid were re-suspended in PBS and preserved at 4° C. for subsequent use.

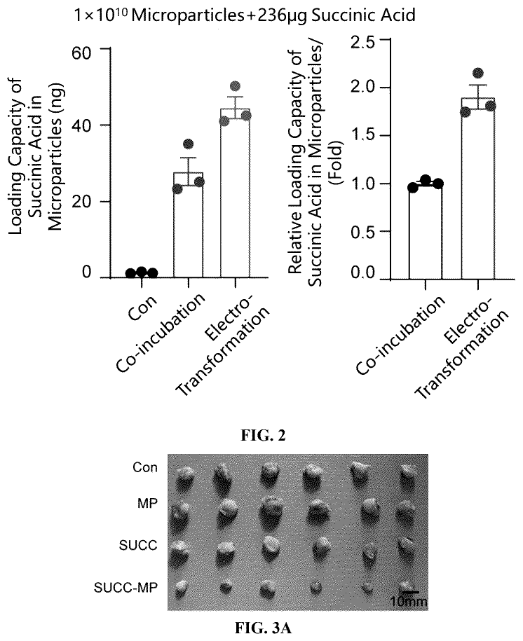

The results were as shown in FIG. 2. Compared with the situation where succinic acid was directly added and then co-incubated with tumor cells (the co-incubation was carried out according to the following specific steps: 1×10⁷ tumor cells were mixed with 1 mM succinic acid; after the mixture was incubated at 37° C. for 24 h, the cells were collected and centrifuged at 800 g for 5 min; the supernatant was collected and subjected to gradient centrifugation at 1000 to 50000 g for 1 to 1.5 h, where the specific steps of the gradient centrifugation were as same as those described in Example 1; the resulting pellets were the tumor cell microparticles loaded with succinic acid), the concentration of succinic acid in the tumor cell microparticles obtained by the electroporation method was higher. Thus, the electroporation method could better improve the succinic acid-loading capacity of tumor cell microparticles.

### Example 3: Therapeutic Effects of Succinic Acid-Loaded Tumor Cell Microparticles on Mouse Subcutaneous Tumor Model of Breast Cancer, Mouse Colorectal Cancer Model, and Mouse Subcutaneous Tumor Model of Liver Cancer

3×10⁵ 4T1 mouse breast cancer cells were subcutaneously inoculated into BALB/c mice to construct mouse subcutaneous tumor models of breast cancer. 3×10⁵ CT26 mouse colorectal cancer cells were intraperitoneally injected into BALB/c mice to construct mouse colorectal cancer models. 3×10⁵ H22 mouse liver cancer cells were subcutaneously inoculated into BALB/c mice to construct mouse subcutaneous tumor models of liver cancer. One week later, treatment was started by intraperitoneally injecting microparticles and drugs into each of the model mice. Each of the models were divided into four groups respectively (5 to 6 mice in each group): a blank control group Con, a microparticle group MP (1×10¹¹ microparticles/mouse), a succinic acid group SUCC (550 ng/mouse), and a succinic acid-loaded microparticle group SUCC-MP (1×10¹¹ microparticles/mouse, corresponding to the succinic acid at 550 ng/mouse). The mice were dosed every two days for two consecutive weeks of treatment. The subcutaneous tumors in mice were observed and the volumes were measured.

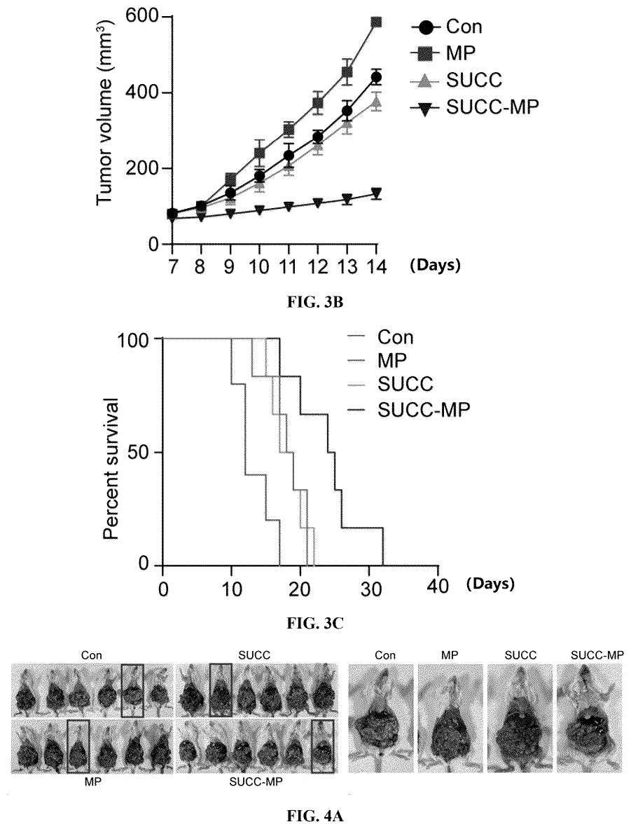

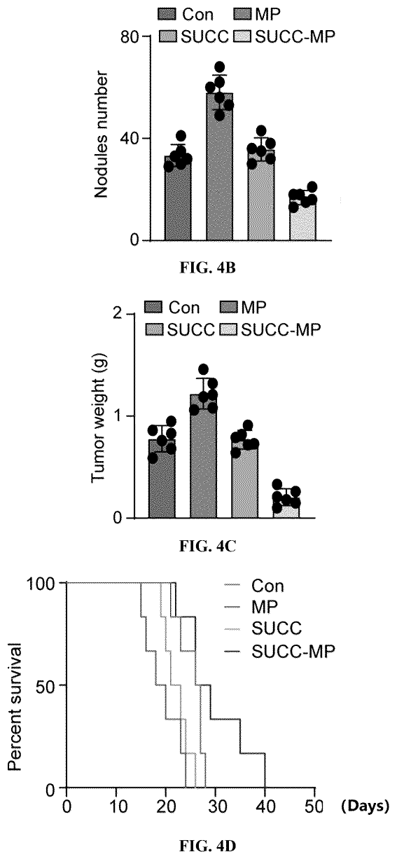

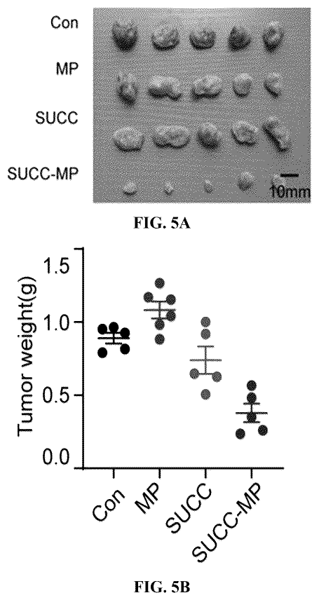

The results were as shown in FIG. 3A to FIG. 5D, showing the therapeutic effects of the succinic acid-loaded tumor cell microparticles on the mouse tumor models. FIGS. 3A and 3B showed that after treatment, the tumor volume became smaller in the mouse subcutaneous tumor model of breast cancer. The tumors shown in FIG. 3A were obtained on the same day after treatment for two weeks. FIG. 3C showed the prolonged survival time of mice after treatment. FIGS. 4A and 4B showed a decrease in abdominal tumor nodules of the mouse colorectal cancer model after treatment for two weeks. FIG. 4C showed a decrease in the tumor weight of mouse colorectal cancer after treatment for two weeks. FIG. 4D showed the prolonged survival time of mice after treatment. FIGS. 5A, 5B, and 5C showed that the tumor volume became smaller and the tumor weight was decreased in the mouse subcutaneous tumor model of liver cancer after treatment. The tumors shown in FIG. 5A were obtained on the same day after treatment for two weeks. FIG. 5B showed the statistical results after treatment for two weeks. FIG. 5D showed the prolonged survival time of mice after treatment. The results suggested that the succinic acid-loaded tumor microparticles achieved therapeutic effects on the mouse breast cancer model, the mouse colorectal cancer model, and the mouse liver cancer model.

### Example 4: Uptake of Tumor Cell Microparticles by Different Immune Cells

The spleens were isolated from C57/bl mice and ground thoroughly. Red blood cells were lysed, and the cell pellets were collected. The cells were plated at 1×10⁶ cells/mL in a 6-well plate, with 1×10⁵ tumor cell microparticles added into each well. The plate was placed in a cell incubator at 37° C. and cultured for 0.5 h and 2 h. The cells were collected. The microparticles were stained with PKH-26. Uptake of microparticles by different immune cells was detected by flow cytometry.

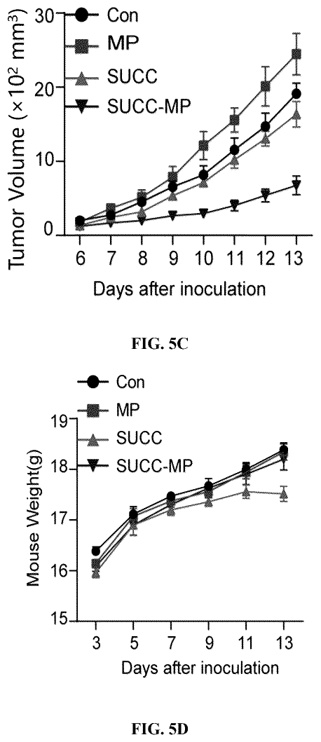

The results were as shown in FIG. 6A and FIG. 6B. FIG. 6A showed that macrophages had the strongest ability to ingest the tumor cell microparticles. FIG. 6B showed that macrophages ingested the most tumor cell microparticles at 2 h. The results suggested that the macrophages could ingest the tumor cell microparticles.

### Example 5: Co-Localization of Macrophages and Tumor Cell Microparticles

From the mouse subcutaneous tumor model of liver cancer constructed in Example 3, the mouse subcutaneous tumor was peeled off at the end of treatment, frozen, and sliced. The tissue to be stained was circled with a Hydrophobic Barrier Pen and permeabilized with 0.5% TritonX-100 (formulated in PBS) at room temperature for 20 min. The slices were blocked with PBS containing 10% BSA at room temperature for 1 h. Flow cytometry antibodies F4/80 were added to label the macrophages, and the tumor cell microparticles were labeled with PKH-26 (1:100) and incubated at 4° C. for 1 h in the dark. The cell nuclei were labeled with DAPI (1:1000) and washed three times with PBS, 5 min for each time. The slices were blocked with an antifade mounting medium and detected by a laser confocal microscope.

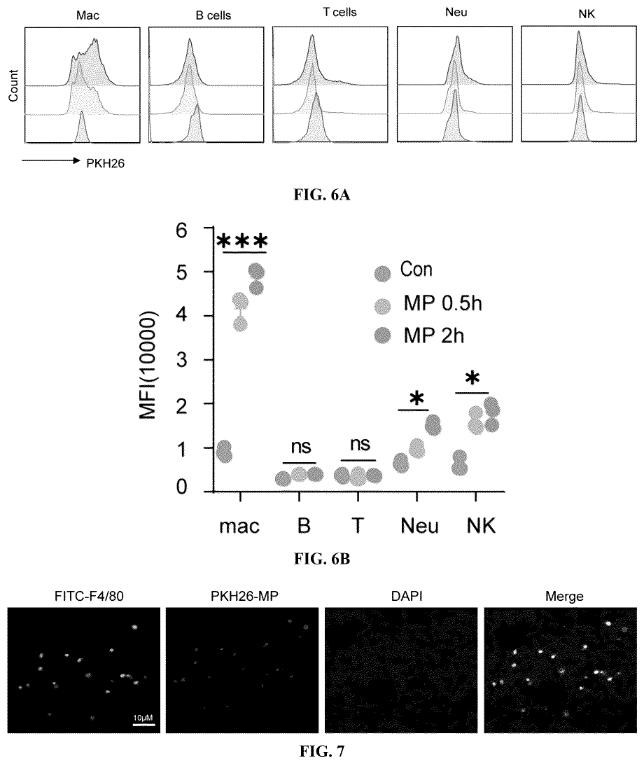

The results were as shown in FIG. 7. Macrophages co-localized with the tumor cell microparticles. The results suggested that the succinic acid-loaded tumor microparticles might exert their antitumor effects as a result of their uptake by macrophages.

1. A tumor cell-derived microparticle loaded with succinic acid, comprising a tumor cell-derived microparticle and succinic acid loaded therein.

2. The tumor cell-derived microparticle loaded with the succinic acid according to claim 1, wherein the tumor cell is a solid tumor cell or a nonsolid tumor cell; optionally, the tumor cell-derived microparticle has a particle size of 50 to 500 nm.

3. The tumor cell-derived microparticle loaded with the succinic acid according to claim 1, wherein a ratio of a number of the tumor cell-derived microparticle to a content of the succinic acid is 1×10⁹ to 1×10¹¹: 25 to 60 ng.

4. A method for preparing a tumor cell-derived microparticle loaded with succinic acid, comprising the following steps:
(i) inducing apoptosis of a tumor cell to release a tumor cell-derived microparticle, mixing the tumor cell-derived microparticle with succinic acid to obtain a mixture I, and subjecting the mixture I to electro-transformation and centrifugation to obtain the tumor cell-derived microparticle loaded with the succinic acid; or
(ii) mixing a tumor cell with succinic acid to obtain a mixture II, co-incubating the mixture II, inducing apoptosis of the tumor cell in the mixture II to release a tumor cell-derived microparticle loaded with the succinic acid, and obtaining the tumor cell-derived microparticle loaded with the succinic acid after centrifugation.

5. The method according to claim 4, wherein a number of the tumor cell in the mixture II is 1×10⁷ to 1×10⁸; a number of the tumor cell-derived microparticle in the mixture I is 1×10¹⁰ to 1×10¹¹; and the succinic acid in the mixture I or the mixture II has a concentration of 0.5 to 2 mM.

6. The method according to claim 4, wherein the inducing apoptosis of the tumor cell is conducted under conditions of suspending the tumor cell in normal saline and leaving at 37° C. for 20 to 30 h; and the centrifugation is conducted under conditions of centrifuging at 500 to 50000 g for 1 to 1.5 h.

7. The method according to claim 4, wherein the electro-transformation is conducted under the following conditions: 150 to 300 V of voltage; 100 to 200 μF of capacitance; 3 to 5 ms of pulse time; and 1 to 6 times of electroporation.

8. The method according to claim 4, wherein the co-incubation is conducted under conditions of leaving at 37° C. for 20 to 30 h.

9. A method for preventing and/or treating cancer, wherein the method comprises administering to a subject a therapeutically effective amount of the tumor cell-derived microparticle loaded with the succinic acid according to claims 1

10. The method according to claim 9, wherein the cancer comprises liver cancer, bladder cancer, bone cancer, brain cancer, breast cancer, colorectal cancer, endometrial cancer, cervical cancer, esophageal cancer, eye cancer, head and neck cancer, kidney cancer, lung cancer, gallbladder cancer, melanoma, ovarian cancer, pancreatic cancer, prostate cancer, gastric cancer, thyroid cancer, leukemia, lymphoma, and myeloma.

11. The tumor cell-derived microparticle loaded with the succinic acid according to claim 2, wherein a ratio of a number of the tumor cell-derived microparticle to a content of the succinic acid is 1×10⁹ to 1×10¹¹: 25 to 60 ng.

12. The method according to claim 5, wherein the inducing apoptosis of the tumor cell is conducted under conditions of suspending the tumor cell in normal saline and leaving at 37° C. for 20 to 30 h; and the centrifugation is conducted under conditions of centrifuging at 500 to 50000 g for 1 to 1.5 h.

13. The method according to claim 5, wherein the electro-transformation is conducted under the following conditions: 150 to 300 V of voltage; 100 to 200 μF of capacitance; 3 to 5 ms of pulse time; and 1 to 6 times of electroporation.

14. The method according to claim 5, wherein the co-incubation is conducted under conditions of leaving at 37° C. for 20 to 30 h.

15. A method for preventing and/or treating cancer, wherein the method comprises administering to a subject a therapeutically effective amount of the tumor cell-derived microparticle loaded with the succinic acid prepared by the method according to claim 4.
